(19) United States
(12) Patent Application Publication
(10) Pub. No.: US 2022/0249717 A1
(43) Pub. Date: Aug. 11, 2022

(54) DEVICES AND METHODS FOR CLEANING ARTICLES WITH ULTRAVIOLET LIGHT

(71) Applicant: Archimedes Innovations, PBC, Ann Arbor, MI (US)

(72) Inventors: Peter Maine Forhan, Ann Arbor, MI (US); Margarita Hernandez, Ann Arbor, MI (US)

(21) Appl. No.: 17/533,785

(22) Filed: Nov. 23, 2021

Related U.S. Application Data
(63) Continuation of application No. PCT/US20/34542, filed on May 26, 2020.
(60) Provisional application No. 62/853,576, filed on May 28, 2019.

(51) Int. Cl. A61L 2/10; A61L 2/20

(52) U.S. Cl. CPC A61L 2/10; A61L 2/202; A61L 2202/14; A61L 2202/11; A61L 2202/26; A61L 2202/122

(57) ABSTRACT

A cleaning device includes an enclosure, an ozone-generating light source, a UVC germicidal light source, and a control system. The ozone-generating light source is configured to generate ozone in the enclosure. The UVC germicidal light source configured to emit UVC germicidal light in the enclosure. The control system is configured to, during a predetermined cleaning cycle, operate the ozone-generating light source for a first time period and to operate the UVC germicidal light source for a second time period, the second time period being at least 10 times longer than the first time period and terminating after the first time period.

### CROSS-REFERENCE TO RELATED APPLICATION(S)

This application is a continuation of PCT Application No. PCT/US2020/034542, filed May 26, 2020, which claims priority to and the benefit of U.S. Provisional Application No. 62/853,576, filed May 28, 2019, the entire disclosures of which are hereby incorporated by reference.

### TECHNICAL FIELD

The present disclosure generally relates to devices and methods for the cleaning of household and other articles and, in particular, devices and methods for cleaning articles using ultraviolet light and/or ozone.

### BACKGROUND

The washing of clothing is a ubiquitous task. Washers and dryers have long been and remain a staple in households of developed countries throughout the world. However, these traditional devices and methods for the cleaning of laundry are financially expensive, resource (water, electricity, natural gas, etc.) intensive and inefficient, time consuming, and potentially harmful to the environment.

For example, the typical cost of standard washing machines designed for household use can range from several hundred to more than one thousand dollars. It is estimated that household washing of laundry accounts for up to 40% of the overall water consumption in a typical household. Older washers use approximately 40-50 gallons of water per load, while newer high-efficiency washers reduce that water demand but still typically require 15-25 gallons of water per load. The purchase and use of harsh chemicals and expensive detergents are most often required to clean clothes in the traditional manner, and these chemicals and detergents are disposed with the waste water produced during the washing process. Washing machines also draw operating electricity, such as to move an agitator or rotate a tub therein often for long periods of time, and copious natural gas is often required to heat the water used in the clothes washing cycle. Moreover, because clothes are typically made wet during cleaning, an expensive and energy demanding dryer is required to return the clothes to dry and wearable condition. It will typically require more than two hours to complete a single load of laundry.

Therefore, there exists a heretofore unmet need in the art for devices and methods for cleaning laundry that restores dirty laundry to a clean state while reducing the cost, natural resource demand, and time required to complete the laundry cleaning process.

### SUMMARY

Disclosed herein are cleaning devices. To resolve the aforementioned unmet need in the art, novel and inventive devices and methods for the cleaning of articles are described herein. More specifically, the present disclosure relates devices and methods for cleaning laundry that use ultraviolet light, may use ozone, and may be waterless. The devices and methods of the present disclosure may be highly effective, simple to construct, and easy to use. They may not require any harsh chemicals, detergents, or natural gas normally required for the proper function of traditional washers and dryers. Moreover, the devices may conserve potable water and public resources for the cleaning of wastewater.

An embodiment of the present disclosure is a cleaning device for the cleaning and sanitization of common household articles, particularly clothing and apparel. It is contemplated that the device may clean a wide array of articles other than those typically found in a household, such as medical equipment, including masks. The cleaning device includes a frame, a shell, an ultraviolet light, and a control system.

The frame includes a supporting architecture for the shell, which may envelope the frame and define an interior of the cleaning device. The frame may further include a rod configured for the hanging of clothing thereon. The frame may have a rectangular cuboid shape configured to stand upright on a surface, similar to the shape of an enclosed, truncated telephone booth.

The shell may include a plurality of layers. A first layer of the shell may be formed of a durable material for the outermost protection of the cleaning device. A second layer of the shell may be formed of an insulating material that is impenetrable by ultraviolet C (“UVC”) light. Suitable materials of the second layer are black nylon and black canvas. A third layer of the shell may be formed of a material suitable for the reflection of ultraviolet light, such as aluminum and/or polyester film including Mylar® film.

At least one or a plurality of UVC light sources are provided within the interior of the cleaning device.

The control system may include one or more power source connectors, switches, processors, data storage means, user interfaces, and communication means, such as wifi and short-range wireless communications technology (i . e., Bluetooth® technology).

In one implementation, a cleaning device includes an enclosure, an ozone-generating light source, a UVC germicidal light source, and a control system. The ozone-generating light source is configured to generate ozone in the enclosure. The UVC germicidal light source configured to emit UVC germicidal light in the enclosure. The control system is configured to, during a predetermined cleaning cycle, operate the ozone-generating light source for a first time period and to operate the UVC germicidal light source for a second time period, the second time period being at least 10 times longer than the first time period and terminating after the first time period.

The cleaning device may further include fan that is operated by the control system during the predetermined cleaning cycle. The first time period may be between 10 seconds and 1 minute and/or be configured to generate between 2.5 ppm and 10 ppm of ozone in the enclosure if the enclosure were to not contain articles to be cleaned. The second time period may be between 5 minutes and 20 minutes, be configured relative to the first time period to substantially deplete the ozone in the enclosure, and/or start one of simultaneous with the first time period or after starting the first time period.

In one implementation, a cleaning device includes an enclosure, an ozone-generating light source, a UVC germicidal light source, and an article support. The ozone-generating light source is configured to generate ozone in the enclosure. The UVC germicidal light source is configured to emit UVC germicidal light in the enclosure. The article is configured to suspend an article therefrom within the enclosure to be cleaned by the ozone and the UVC germicidal light.

The cleaning device may further include a fan that is operable to circulate gas within the enclosure to move the articles. The article support may include one or more lines that are flexible suspended from an upper portion of the enclosure and/or arranged at lateral spacing of at least 6 inches. The article support may further include attachment devices that are configured to releasably couple to the articles to be cleaned and that may be spaced apart along the lines at vertical spacing of at least 6 inches. The enclosure may include four upright sides that are flexible, supported by a frame, and/or include an inner layer that forms reflects the UVC germicidal light in the enclosure.

In one implementation, a method for cleaning face masks with a cleaning device includes: receiving face masks in an enclosure of the cleaning device; supporting the face masks in the enclosure with an article support of the cleaning device at predetermined locations on the support that are spaced apart by at least 6 inches; generating ozone in the enclosure with an ozone-generating light source of the cleaning device; and emitting UVC germicidal light in the enclosure with a UVC germicidal light source of the cleaning device.

The method may further include emitting the UVC germicidal light for a predetermined time period configured for the ozone to be substantially depleted from the enclosure. The method may further include operating a fan to circulate the ozone and to move the face masks within the enclosure.

The devices disclosed herein may clean and disinfect articles, such as clothing, using UVC lights and, thus, may advantageously obviate or dramatically reduce the need for traditional washers and dryers to clean most clothing.

The devices disclosed herein may clean articles, such as clothing, advantageously using essentially no water, natural gas, or detergents, and may require approximately 1% of the total energy required by traditional washers and dryers on a per load basis.

The devices disclosed herein may be lightweight, have a small physical footprint, and be stowed away when not in use.

Examples of the more important features of the disclosure have been summarized rather broadly in order that the detailed description thereof that follows may be better understood and in order that the contributions they represent to the art may be appreciated. There are, of course, additional features of the disclosure that will be described hereinafter and which will form the subject of the claims appended hereto.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a schematic view of a cleaning device.

FIG. 2A is a perspective view of the cleaning device with a chamber closed by a closure.

FIG. 2B is a perspective view of the cleaning device with the chamber opened by the closure.

FIG. 3 is a method of flow diagram of a method for operating the cleaning device.

FIG. 4A is a perspective view of the cleaning device further illustrating light sources an agitator with hidden components depicted in dashed lines.

FIG. 4B is a perspective view of the cleaning device further illustrating a frame and first variation of an article support with hidden components depicted in dashed lines.

FIG. 4C is a cross-sectional view of the cleaning device taken along lines 4C-4C in FIGS. 4A and 4B.

FIG. 4D is a front view of the cleaning device with the first variation of the article support.

FIG. 4E is a perspective view of the cleaning device with a second variation of the article support in a use state (solid lines) and a stowed state (dash-dot lines).

FIG. 4F is a front view of the cleaning device with the second variation of the article support in the use state and the stowed state.

FIG. 4G is a perspective view of the cleaning device with a third variation of the article support in a use state (solid lines) and a stowed state (dash-dot lines).

FIG. 4H is a front view of the cleaning device with the third variation of the article support in the use state and the stowed state.

FIG. 4I is a perspective view of the cleaning device with a fourth variation of the article support with articles coupled thereto and hidden components depicted in dashed lines.

FIG. 4J is a front view of the cleaning device with the fourth variation of the article support with the articles coupled thereto.

FIG. 5 is a perspective view of a variation of the cleaning device having one or more movable light sources.

FIG. 6 is a front perspective view of a preferred embodiment of the present invention.

FIG. 7 is another front perspective view of the preferred embodiment of the present invention of FIG. 6 having clothing articles provided therein.

FIG. 8 is yet another front perspective view of the preferred embodiment of the present invention of FIG. 6 showing a portion of a control system comprising the invention.

FIG. 9 is an illustration of an alternative preferred embodiment of the present invention.

FIG. 10 is an illustration of another alternative preferred embodiment of the present invention.

### DETAILED DESCRIPTION

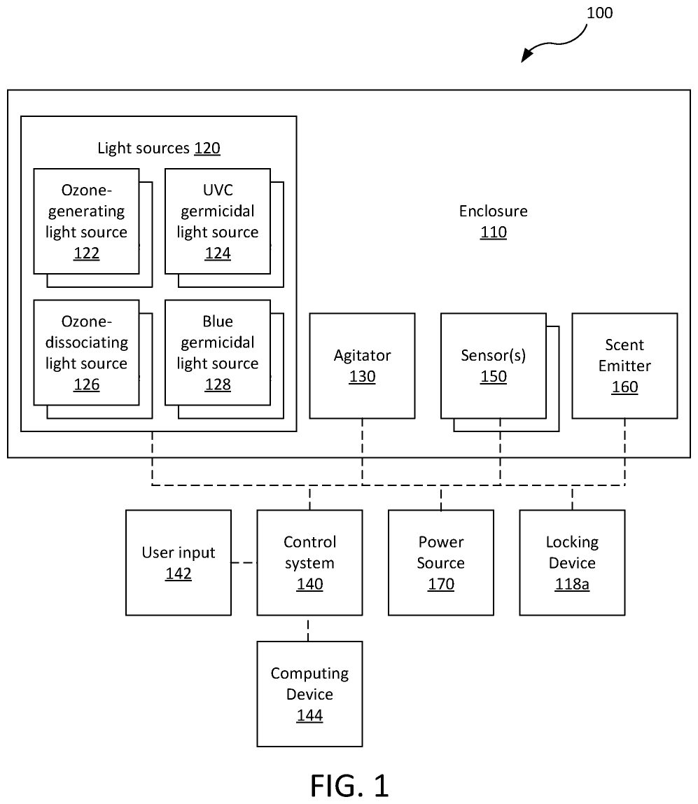

Referring to FIG. 1, a cleaning device 100 is provided for cleaning articles with ozone and/or germicidal light. For example, the cleaning device 100 may be used and may be configured for cleaning articles, such as clothing, medical personal protection equipment, other textile articles, and rigid devices (e.g., electronic devices, such as laptop computers or smartphones). The cleaning device 100, and variations thereof discussed below, each generally include an enclosure 110, one or more light sources 120, a control system 140, and a power source 170. The cleaning device 100 may also include an agitator 130, one or more sensors 150, and/or a scent emitter 160.

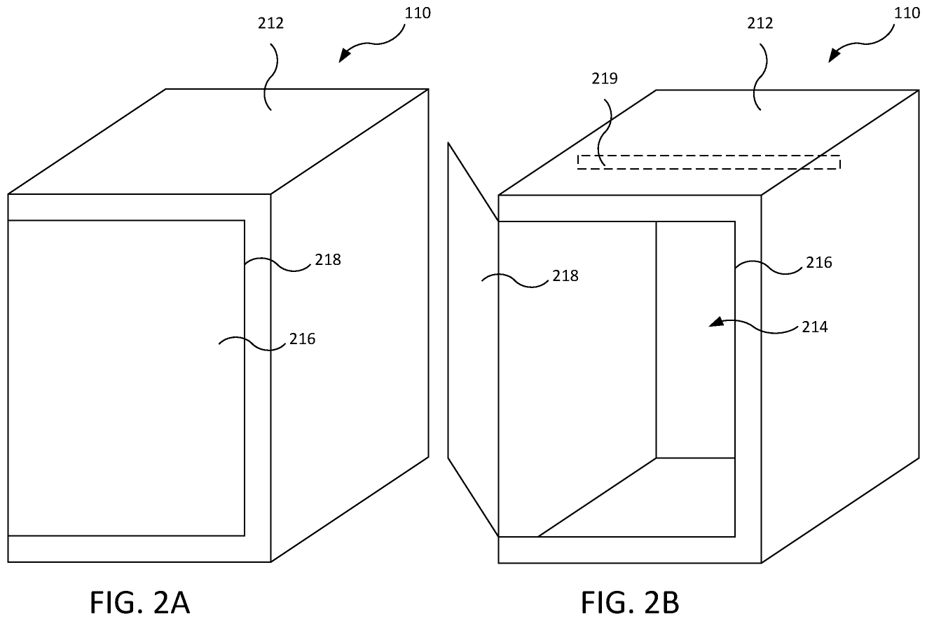

Referring to FIGS. 2A-2B, the enclosure 110 is configured to receive the articles therein to be cleaned. The enclosure 110 may be any suitable size to receive articles therein to be cleaned. In preferred examples, the enclosure 110 is sized to receive clothing therein, for example, having a width of between 1 foot and 6 feet, a depth of between 1 foot and 4 feet, and a height of between 2 feet and 6 feet (e.g., two feet by two feet by four feet), more or less. The enclosure 110 may further be configured to be portable, for example, being sized and or reconfigurable (e.g., collapsible) to be moved through standard doorways between rooms.

The enclosure 110 may also be configured to be sealed to hinder (e.g., prevent) exfiltration of gases from the enclosure 110, such as ozone generated therein. The enclosure 110 generally includes a body 212 (e.g., a shell) that defines a chamber 214 (e.g., an interior) and an opening 216 through which the articles are received into and removed from the chamber 214. The body 212 further includes a closure 218, such as a door, lid, or other structure, which is movably coupled and/or releasably coupleable to the body 212 to close the opening 216. The closure 218 may additional seal the opening 216 to hinder (e.g., prevent) the exfiltration of gases and/or leakage of potentially harmful light from the chamber 214. The body 212 of the enclosure may further include interior surfaces that are reflective to light emitted by the light sources 120, such as UVC germicidal light, to reflect the light impinge on different sides of the articles being cleaned. The enclosure 110 may further include an article support 219 that is arranged within the chamber 214 and configured to support (e.g., suspend) the articles within the chamber 214 and out of contact with the body 212 for cleaning with the ozone and/or light.

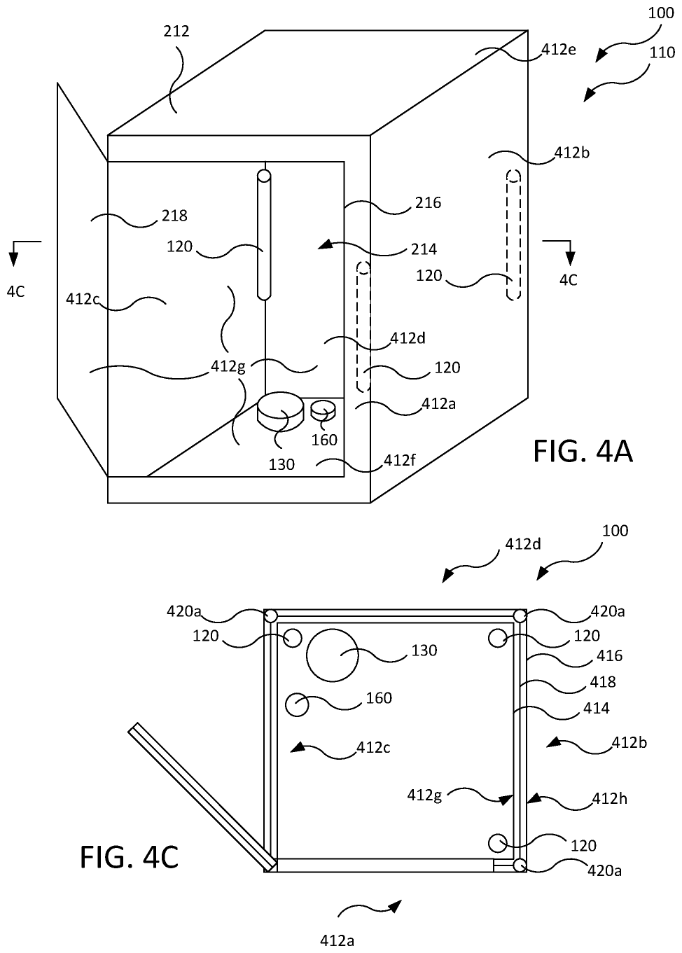

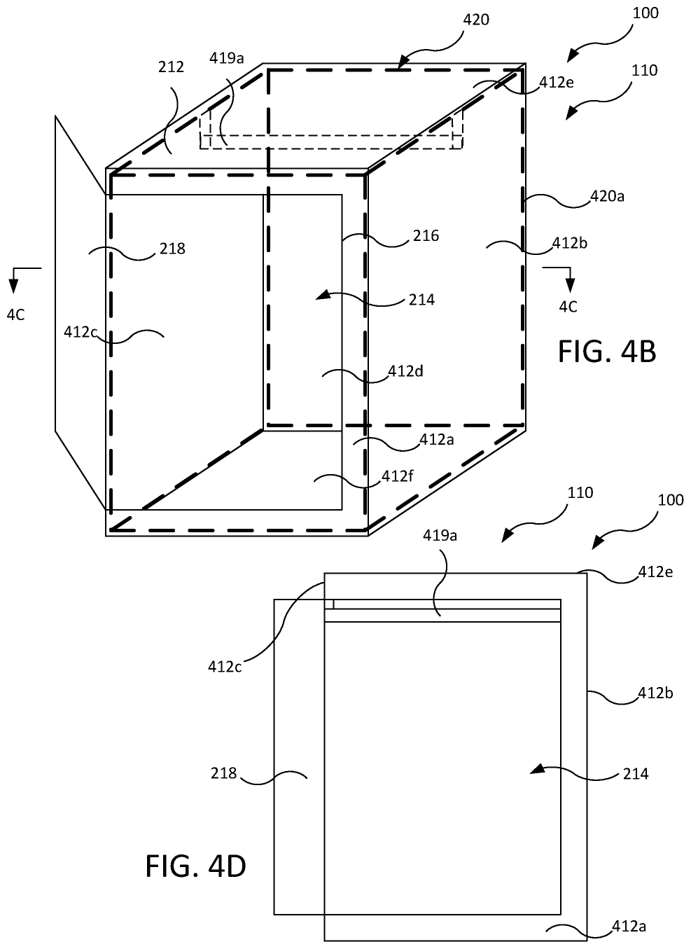

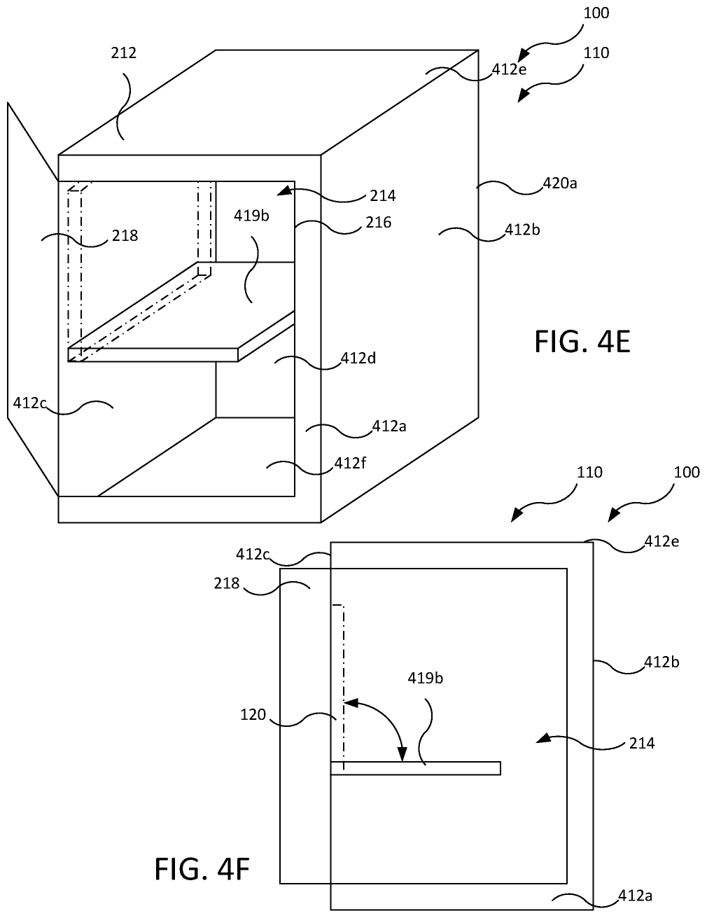

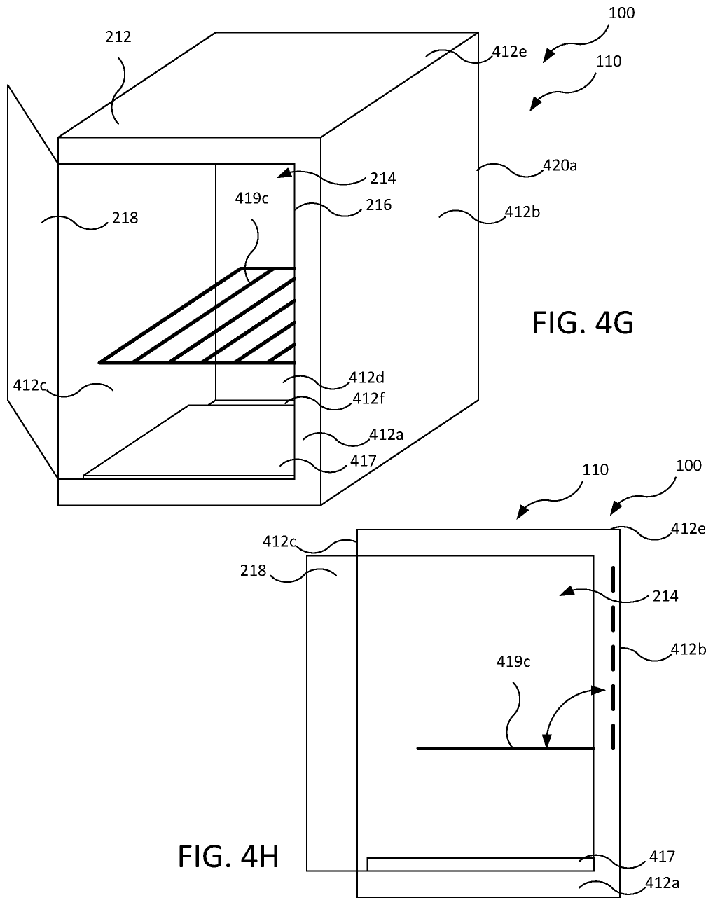

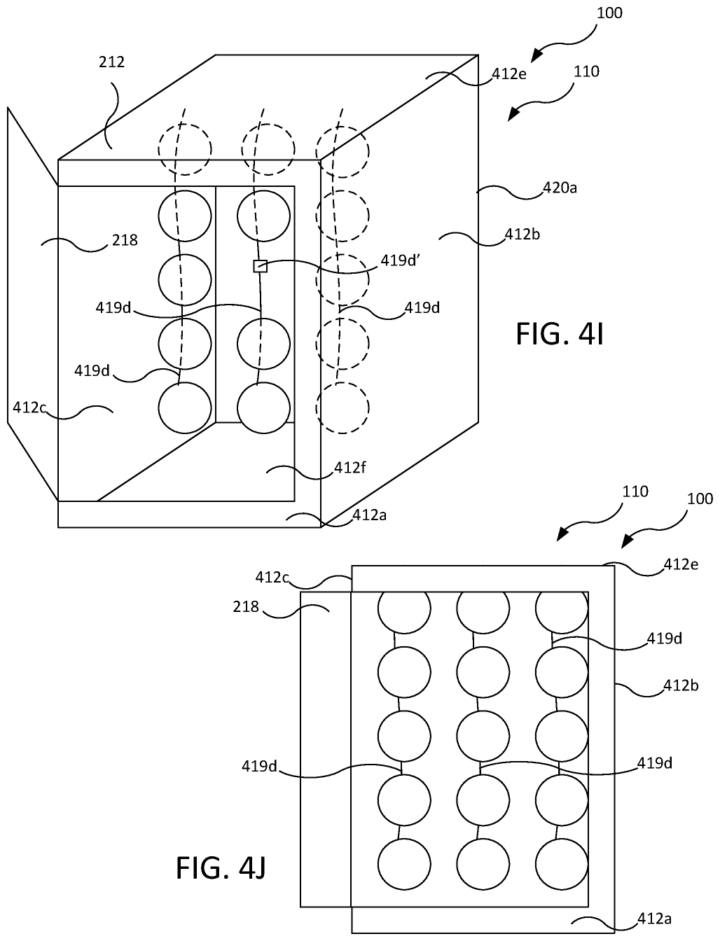

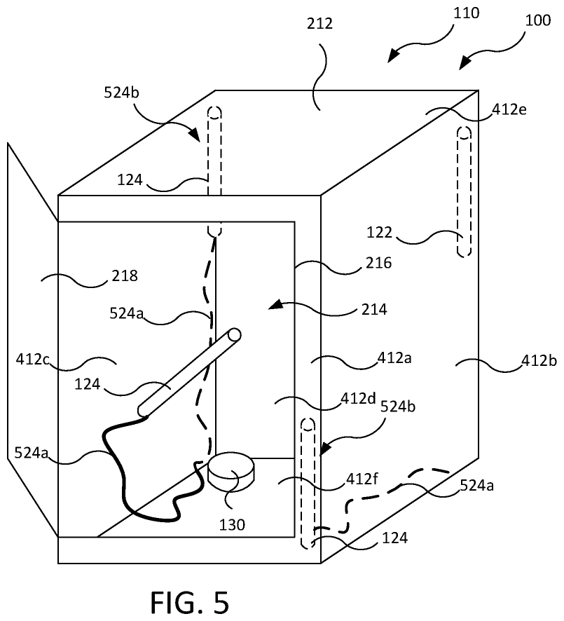

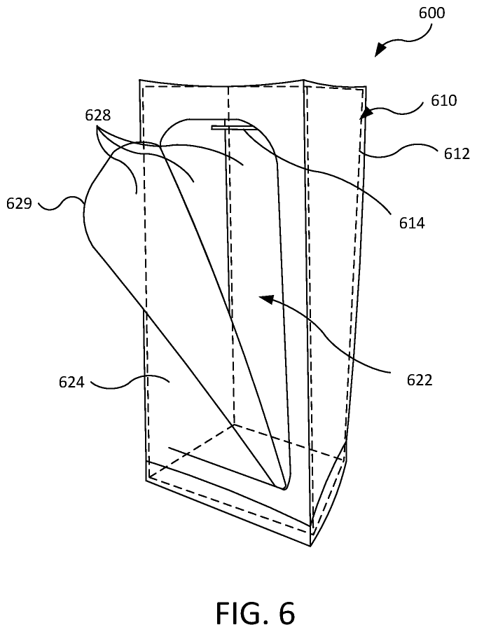

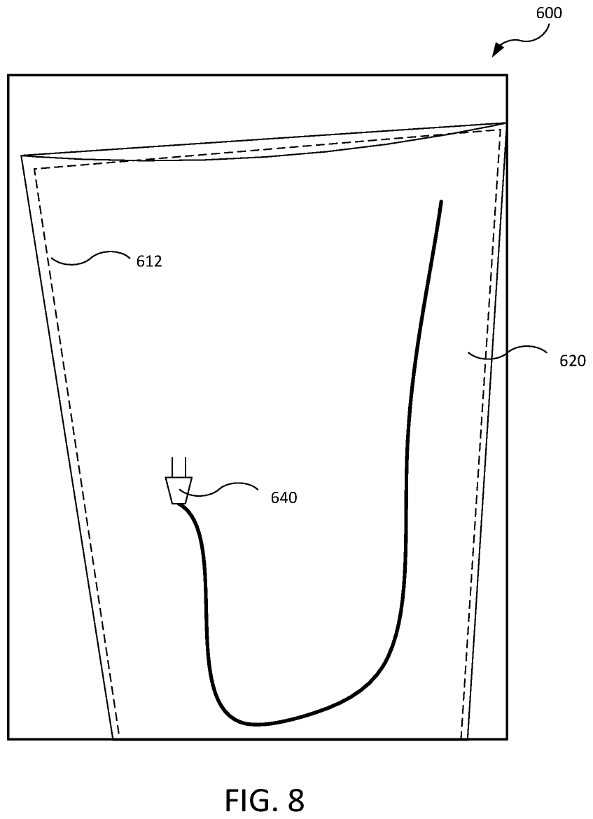

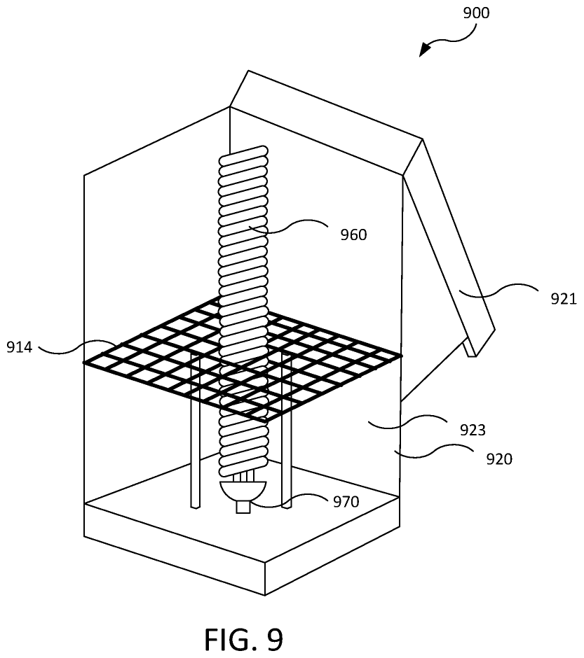

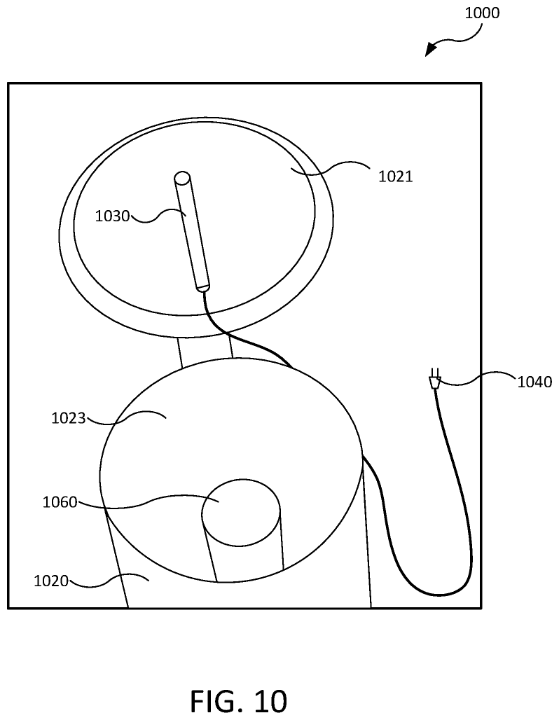

Further aspects and configurations of the cleaning device 100, the enclosure 110, and variations thereof are discussed in further detail below. For example, the enclosure 110, the light sources 120, and/or the article support 219 may be as described below with respect to FIGS. 4A-5, with respect to FIGS. 6-8 (e.g., the enclosure 110 and body 212 being cooperatively formed by the frame 610 and the shell 620, the chamber 214 being formed by the interior 622, the closure 218 being formed by the door 629, the article support 219 being formed by the hanging rod 614), with respect to FIG. 9 (e.g., the enclosure 110 and the body 212 being cooperatively formed by the base 914 and the shell 920, the closure 218 being formed by the lid 921), and/or with respect to FIG. 10 (e.g., the enclosure 110 and the body 212 being cooperatively formed by the base 914 and the shell 1020, and the closure 218 being formed by the 921).

The closure 218 may further be configured to prevent access to the chamber 214 during a cleaning cycle or in other circumstances. For example, the closure 218 may include a locking device 118a that prevents moving the closure 218 to unseal the opening 216 during a cleaning cycle, for example, to prevent release of ozone from the chamber 214. The locking device 118a may, for example, be a magnetic or mechanical latch. The locking device 118a may further include or be configured as a sensor that detects whether the closure 218 is opened or to communicate a state thereof (e.g. locked or unlocked), whereby the control system 140 may further prevent initiation of a cleaning cycle or may stop a cleaning cycle if the locking device 118a is opened or the closure 218 is opened or the locking device 118a is in the unlocked state.

Referring again to FIG. 1, the one or more light sources 120 are configured to clean the articles placed in the enclosure 110. The one or more light sources 120 are configured to one or more of generate ozone, dissociate ozone, or destroy pathogens (e.g., bacteria and viruses) by outputting light in the ultraviolet spectrum and, in some embodiments, by also outputting light in the visible light spectrum. The light sources 120 of the cleaning device include various combinations of one or more of an ozone-generating light source 122, a UVC germicidal light source 124, an ozone-dissociating light source 126, or a blue germicidal light source 128. As an alternative to the ozone-generating light source 122, the cleaning device 100 may include another type of ozone-generating device, such as a corona discharge device (e.g., ozone plate or corona discharge tube), which may output ozone to higher concentrations and/or at faster rates than the ozone-generating light source 122 and, thereby, be operate for lower amounts of time (e.g., between 5% and 20% of the ozone-generating time period discussed below) to achieve the same concentration of ozone.

In the description and claims that follow, the different light sources may also be identified numerically (e.g., a first light source, a second light source, etc.) and/or by the bandwidth of the light emitted therefrom (e.g., a 180-190 nm light source, a 185 nm light source, a UVC light source, an ultraviolet light, etc.). The one or more light sources 120 may also be configured as described below with respect to FIGS. 6-10 (e.g., as the ultraviolet lights 630, 1030).

The ultraviolet spectrum of light includes ultraviolet A (“UVA”) light, ultraviolet B (“UVB”) light, and ultraviolet C (“UVC”) light. UVA light typically has wavelengths of 315-400 nanometers (“nm”) and, for example, is associated with the “tanning” of human skin due to sun exposure, for example. UVB light typically has wavelengths of 280-315 nm and, for example, is associated with sunburn due to sun exposure. UVC light typically has wavelengths of 100-280 nm.

The ozone-generating light source 122 emits UVC light that generates ozone. Ozone may oxidize with and, thereby, kill or otherwise destroy bacteria and other organic matter. Human exposure to ozone at high concentrations and/or extended periods of time may have harmful effects.

UVC light generates ozone at wavelengths below 200 nm with peak ozone generation occurring at between 180 nm and 190 nm (e.g., at 185 nm). UVC light destroys ozone at wavelengths above 220 nm. UVB light also dissociates ozone at wavelengths below 315 nm with peak effect between 285 nm and 295 nm.

The ozone-generating light source 122 may emit UVC light with a peak wavelength of between 180 nm and 190 nm such as at 185 nm, which may be referred to as ozone-generating light. The ozone-generating light source 122 may also emit UVC light that dissociates ozone, such as with another peak wavelength of between 250 nm and 265 nm, such as 254 nm. To the extent that the ozone-generating light source 122 may emit light that dissociates ozone, the ozone is dissociated at a significantly lower rate than at which the ozone is generated by the ozone-generating light source 122. Thus, the ozone-generating light source 122 is a net producer of ozone. The ozone-generating light source 122 is or includes, in one example, a mercury lamp having an input wattage of between 10 watts and 50 watts (e.g., 15 watts to 25 watts) having one or more tubes (e.g., four), but may be or include any other suitable type of light source (e.g., one or more LEDs). In one specific example, the ozone-generating light source 122 is a 15 watt mercury lamp having a peak output wavelength of 185 nm.

The UVC germicidal light source 124 emits UVC light having germicidal effect. UVC light is a known germicide that kills or destroys microbes, microorganisms, bacteria, viruses, other pathogens, and associated odors, for example, by disrupting chemical bonds in the deoxyribonucleic acid (“DNA”). UVC light has varying germicidal effect at different wavelengths for different pathogens. UVC light may have peak germicidal effect at higher wavelengths, such as between 250 nm and 280 nm, including between 250 nm and 265 nm (e.g., at 254 nm). UVC light may have germicidal effect at lower wavelengths, such as those wavelengths at which ozone is generated.

The UVC germicidal light source 124 may emit UVC light with a peak wavelength of between 250 and 265 nm, such as at 254 nm, which may be referred to as UVC germicidal light. The UVC germicidal light also functions to dissociate ozone, such that the UVC germicidal light source 124, which emits no or negligible ozone-generating light, is a net reducer of ozone. The UVC germicidal light source 124 is or includes, in one example, a mercury lamp having an input wattage of between 10 watts and 50 watts (e.g., 15 watts to 25 watts) having one or more tubes (e.g., four), but may be or include any other suitable type of light source (e.g., one or more LEDs). In one specific example, the UVC germicidal light source 124 is a 15 watt mercury lamp having a peak output wavelength of 254 nm.

The ozone-dissociating light source 126 emits light that dissociates ozone. Ultraviolet light with wavelengths above the UVC spectrum may also dissociate ozone and at a faster rate and/or more efficiently than UVC germicidal light. The ozone-dissociating light source 126 may, for example, emit UVB light with a peak wavelength below 315 nm, such as between 285 nm and 295 nm, such as 290 nm. The ozone-dissociating light source 126 may dissociate ozone at a faster rate and/or more efficiently than the UVC germicidal light. The ozone-dissociating light source 126 is or includes, in one example, a mercury lamp, but may be or include any other suitable type of light source (e.g., one or more LEDs).

The blue germicidal light source 128 emits visible light having germicidal effect. Visible blue light with a peak wavelength of 405 nm at high intensity (e.g., 5-15 J/cm\hat{ }2) has been shown to have germicidal effect. The blue germicidal light source 128 may be visible light with a peak wavelength of between 400 nm and 440 nm. The blue germicidal light source 128 is or includes, in one example, a mercury lamp, but may be or include any other suitable type of light source (e.g., one or more LEDs).

The agitator 130 is configured to move articles being cleaned in the enclosure 110 to provide greater exposure to the light emitted by the light sources 120 and/or to the ozone. In one example, the agitator 130 is a motorized device that directly engages and moves the articles or the article support 219 from which articles are supported within the enclosure 110. In another example, the agitator 130 is a fan that circulates air within the enclosure 110, which in turn moves the articles within the enclosure. In the case of the light sources 120 including the ozone-generating light source 122, providing the agitator 130 as a fan has the added advantage of circulating the ozone within the chamber 214 of the enclosure 110 to provide greater exposure of the articles to the ozone and, thereby, provide greater cleaning effect.

The agitator 130 may be configured as described below with respect FIGS. 6-10 FIGS. 9 and 10 (e.g., being formed cooperatively by the shafts 960, 1060 and the motor 970).

The one or more sensors 150 of the cleaning device 100 may be used to control various operations of the cleaning device 100, for example, preventing operation of the cleaning device 100 or providing feedback for variably controlling operation of the cleaning device 100. The sensors 150 may be provided in the enclosure 110 (as shown in FIG. 2) and/or may be provided exterior thereto. The sensors 150 may include one more of an ozone sensor, a locking sensor, or a weight sensor. With an ozone sensor, the control system 140 may, for example, control ozone generation until a desired concentration of ozone is achieved, control ozone dissociation and/or the locking device 118a until ozone is substantially depleted (as described below), and/or prevent operation of the cleaning device 100 (e.g., ozone generation) if ozone is detected outside the chamber 214 above a threshold. With the locking sensor (e.g., of the locking device 118a) may be used to prevent operation of the cleaning device 100 unless the closure 218 is closed and/or the locking device 118a is in the locked state. In one example, the closure sensor is a switch or relay, which is in an open state if the opening 216 is not closed by the closure 218 to prevent activation of the light sources 120, and which is in a closed state if the opening 216 is closed by the closure 218 to permit activation of the light sources 120. With the weight sensor, the control system 140 may not operate the cleaning device 100, for example, if weight exceeds a threshold (e.g., indicating an inappropriate object being included in the device) or if the weight does not match an expected weight (e.g., for cleaning certain articles).

The control system 140 is configured to operate the light sources 120, as well as the agitator 130, the sensors 150, and the scent emitter 160 if included. The control system 140 may be configured in any suitable manner for operating the cleaning device 100 in the manners described herein. In one example, the control system 140 may include one or more time switches that operate the light sources 120 and the agitator 130 for predetermined amounts of time upon receiving a user input (e.g., pressing a button or other input device of the control system 140). In another example, such as in implementations that include operational sensors, the control system 140 may be a microcontroller or other controller that operates the cleaning device 100 according stored control logic or programming (e.g., having a processing unit, a volatile memory, a storage that includes instructions by which components of the cleaning device 100 are operated, and a communications interface for sending and/or receiving signals to and/or from the other components).

The control system 140 may also include a user input 142 by which the users may provide one or more inputs for operating the cleaning device 100. In one example, the user input 142 includes one or more physical buttons by which the user may initiate a single predetermined cleaning cycle, may select and initiate one of multiple predetermined cleaning cycles, or may input various parameters (e.g., type and quantity of articles to be cleaned and/or level of cleaning desired) according to which the control system 140 determines a cleaning cycle. In a still further example, the user input 142 may be or further include a wireless communications device that is configured to communicate with a user device, such as a computing device 144 (e.g., a smartphone) of a user by which the user may provide any of the foregoing inputs to the cleaning device 100. For example, the user may provide inputs to the computing device 144 to initiate a single predetermined cleaning cycle or select and initiate one of multiple predetermined cleaning cycles. Instead or additionally, the user may input to the computing device 144 other operational parameters, such as user-defined operational time periods for the light sources 120 or result-oriented operations of the light sources (e.g., quantified ozone generation). The computing device 144 may include software programming by which operational parameters are communicated to the user input 142 (e.g., operational time periods of the light sources 120) based on the user input and/or by which the user inputs are constrained (e.g., by limiting user-defined operational time periods of the light sources 120, for example, to limit ozone production and/or to ensure sufficient ozone-dissociation).

The control system 140 may also be configured as described below with respect to FIGS. 6-10 (e.g., as the control system 640, the control system 1040, or variations thereof described below).

The scent emitter 160 is configured to emit a scent (e.g., a fragrance) into the chamber 214. While the articles may be sufficiently cleaned by the ozone and/or germicidal light, emitting scent that is absorbed by the articles may be desirable and/or provide confirmation to the user that the article has been cleaned. The scent emitter 160 may be any suitable type of device, such as heated oil diffuser or aerosol can. Those scents that are emitted from the scent emitter 160 may be selected from chemicals that do not react with ozone to produce formaldehyde or other noxious gases.

The power source 170 is configured to provide electrical power for operation of the various other electronic components of the cleaning device 100 (e.g., the light sources 120, the agitator 130, the control system 140, the sensors 150, and the scent emitter 160). The power source 170 may, for example, power storage device (e.g., a battery) or a wired connection device (e.g., a plug for connecting to another power source, such as conventional wall outlet), along with appropriate circuitry for providing the electrical power for operation the other electronic components. The power source 170 being a power storage device (e.g., a battery) or other portable power supply may be advantageous for use of the cleaning device 100 in regions without reliable sources of electrical power. The power source 170 may also function as part of the control system 140, for example, beginning a cleaning cycle when the power source 170 is begins providing electrical power (e.g., when turned on or when plugged in).

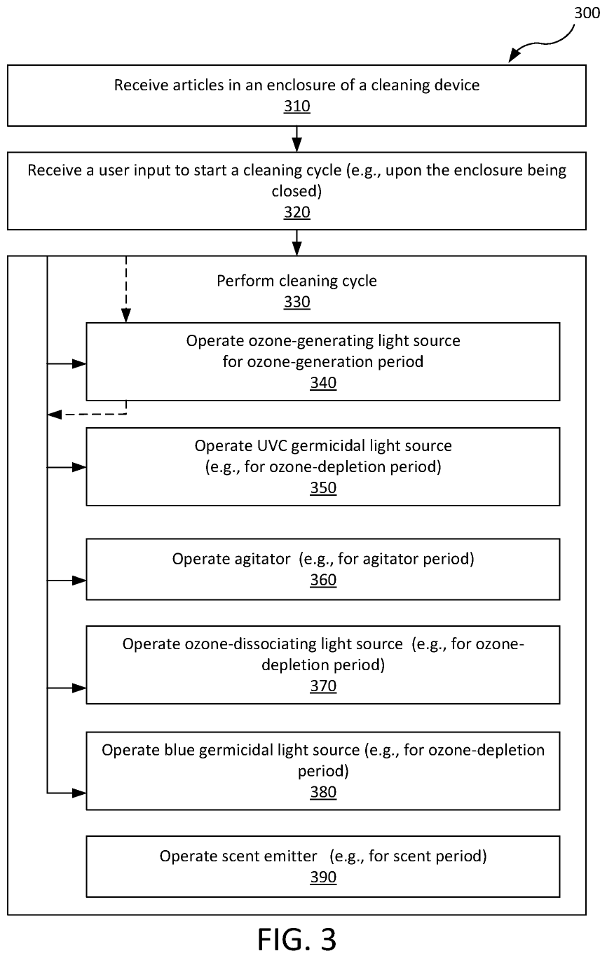

Referring to FIG. 3, different embodiments of the cleaning device 100 may be operated according to a method 300. The user provides an input to start a cleaning cycle during which the ozone-generating light source 122 and the UVC germicidal light source are operated, as well as, if included, the ozone-dissociating light source 126, the blue germicidal light source 128, the agitator 130, and/or the scent emitter 160.

During one or more cleaning cycles, the light sources 120 are operated to clean the articles in the chamber 214. During ozone cleaning cycles, the cleaning device 100 cleans the articles with both ozone and germicidal light, which includes producing and substantially depleting ozone from the chamber 214. During germicidal-only cleaning cycles, the cleaning device 100 cleans articles with germicidal light but not ozone.

During the cleaning cycles, the light sources 120, the agitator 130, and/or the scent emitter 160 may be operated for operational time periods, which are predetermined time periods configured to achieve desired effects, which may include ozone generation, germicidal effect, and ozone depletion. The operational time periods for the light sources 120 may differ between different cleaning cycles as discussed below. As further discussed below, instead of or in addition to operating according to the operational time periods, various of the light sources 120 may be configured to operated according to feedback provided by the sensors 150 (e.g., an ozone sensor).

The ozone-generating light source 122 is operated for a first time period, which may be referred to as the ozone-generating time period, and the UVC germicidal light source 124 is operated for a second time period, which may be referred to as the germicidal time period. If included, the ozone-dissociating light source 126 is operated for an ozone-dissociating time period, the blue germicidal light source 128 is operated for a blue-germicidal time period, the agitator 130 is operated for an agitator time period, and/or the scent emitter 160 is operated for a scent-emitting time period. Operational time periods for each of the foregoing components may also be referred to by a like name (e.g., the ozone-generating light source 122 being operated for an ozone-generating time period) or numerically (e.g., first, second, and third time periods).

As discussed in further detail below, during a cleaning cycle, the operational time periods of the light sources 120 may be started in any suitable order. The ozone-generating time period may be started simultaneous with those other operational time periods, which may provide for simplification of the control system 140 or control logic thereof. The ozone-generating time period also ends prior to at least one of UVC germicidal time period and/or the ozone-dissociating time period, which allows for the UVC germicidal light source 124 and/or the ozone-dissociating light source 126 to dissociate the ozone to substantially deplete the ozone before completing the cleaning cycle.

In an ozone cleaning cycle, such as for cleaning loosely arranged clothing articles, the ozone-generating time period is configured for the ozone-generating light source 122 to generate sufficient ozone for cleaning the article, such as between 2 ppm and 10 ppm, such as between 4 ppm and 8 ppm, or more. While 2.5 ppm of ozone is believed to be a sufficient concentration of ozone to clean loosely arranged clothing articles, including textile articles, higher concentrations of ozone may allow for deeper penetration of the ozone into articles being cleaned, such as a basket of clothing articles. Factors influencing the concentration of ozone generated during the ozone-generating time period include the ozone-generating light source 122 itself (e.g., number, peak wavelength, and/or energy output), a volume of the chamber 214 of the enclosure 110, and, to a lesser extent, environmental conditions (e.g., humidity and temperature). The ozone-generation time period may, for example, be between 5 seconds and 2 minutes, such as between 10 seconds and 60 seconds, or 30 seconds, more or less. For example, with the ozone-generating light source 122 configured as a 15 watt mercury lamp with a peak wavelength at 185 nm and the chamber 214 being sixteen cubic feet (e.g., two feet wide by two feet deep by four feet tall), operating the ozone-generating light source 122 for 30 seconds may generate 8 ppm of ozone if no articles are placed in the chamber 214 with which the ozone can react. The ozone-generation time period be defined by the wattage of the ozone-generating light source 122 and the volume of the chamber 214, for example, being operated between 1 second and 10 seconds (e.g., 2 seconds to 4 seconds) for each cubic foot of volume per 25 watts of power input to the ozone-generating light source 122 (e.g., 50 cubic feet and two 25 watt bulbs, operating between 25 seconds and 250 seconds (e.g., between 50 seconds and 100 seconds).

In another example of an ozone cleaning cycle, such as for a basket of compacted clothing articles, the ozone-generation time period may be longer, so as to create a higher concentration of ozone (e.g., between 10 and 100 ppm), such as between 40 and 80 ppm. For example, the ozone-generating light source 122 may be operated for an ozone-generation time period of between 2 minutes and 10 minutes, such as 4 minutes.

In still further examples of ozone cleaning cycles, the ozone-generating time period may be defined by the user or set according to a user input (e.g., ppm, level of clean desired, etc.), but which may be limited by the cleaning device 100 to prevent excessive concentrations of ozone.

In still further examples of ozone cleaning cycles, for those cleaning devices 100 that include a sensor 150 configured to detect ozone, the ozone-generating light source 122 may be operated until a predetermined or user-defined concentration of ozone is detected with the sensor 150 (e.g., 2 ppm to 10 ppm, such as 8 ppm, more or less), or until the earlier of achieving the ozone-generating time period or the predetermined concentration.

In germicidal-only cleaning cycles, the cleaning device 100 does not include the ozone-generating light source 122, or the cleaning device 100 is configured to operate another cleaning cycle in which the ozone-generating light source 122 is not operated.

The germicidal time period is configured for the UVC germicidal light source 124 to have the germicidal effect of killing or otherwise destroying pathogens on the articles being cleaned (e.g., textiles). For example, a desired germicidal effect may be achieved upon outputting 1.8 J/cm\hat{ }2 or more of the UVC germicidal light to surfaces of the articles being cleaned. Delivery of sufficient UVC germicidal light for the desired germicidal effect may be influenced by the UVC germicidal light source 124 (e.g., peak wavelength, number, and/or energy output), direct or reflected distance from the UVC germicidal light source 124 to those surfaces of the articles being cleaned, and exposure of those surfaces to the UVC germicidal light (e.g., as moved by the agitator 130). For example, with the UVC germicidal light source 124 being configured as a 15 watt mercury lamp with a peak wavelength of 254 nm within the chamber 214 having dimensions of two feet wide by two feet deep by four feet tall, the germicidal effect may be achieved in under one minute (e.g., under 30, 15, 10, or 5 seconds). The germicidal time period may, therefore, be under 30 seconds to achieve the germicidal effect.

In addition to being configured to achieve a germicidal effect, the germicidal time period may be configured for the UVC germicidal light source 124 to substantially deplete the ozone from the chamber 214 of the enclosure 110 (e.g., to substantially 0 ppm, such as 0.1 ppm or 0.2 mg/m\hat{ }3 or less). To substantially deplete the ozone, the germicidal time period terminates after and is longer than the ozone-generating time period, while the germicidal time period may be started simultaneous with the starting of the ozone-generating time period or thereafter (e.g., before or after termination of the ozone-generation time period).

Depletion of the ozone occurs from oxidizing organic material (e.g., on the articles), natural decay (e.g., into ordinary oxygen), and dissociation from the UVC germicidal light source 124, as well as dissociation from the ozone-dissociating light emitted by the ozone-dissociating light source 126 (if provided). Achieving the desired ozone-depleting effect is expected to take significantly longer than achieving the desired germicidal effect (e.g., multiple times longer). In a longest-case scenario with the chamber 214 including no articles to be cleaned and no substances for the ozone to react with, the ozone is expected to be substantially depleted from 8 ppm in fewer than 10 minutes (e.g., between 9 and 10 minutes). As articles are added to the chamber 214, the ozone is expected to be substantially depleted from 8 ppm more quickly (e.g., less than 5 minutes, such as 3.5 minutes or less), as the ozone reacts with organic material on the articles being cleaned. To substantially deplete the ozone without the ozone-dissociating light source 126, the germicidal time period may, for example, be between 9 minutes and 20 minutes, such as between 9 minutes and 12 minutes, more or less, to ensure help ensure the ozone is substantially depleted in the longest-case scenario, which is also sufficient time to achieve the desired germicidal effect.

For higher concentrations of ozone, such as between 40 ppm and 80 ppm, the germicidal time period may be longer for the ozone to be substantially depleted, such as between 40 minutes and 120 minutes, such as 90 minutes.

The germicidal time period may be defined relative to the ozone-generation time period, such as being between 10 and 50 times, such as between 10 and 30 times (e.g., 20 times), more or less, to substantially deplete the ozone.

In those embodiments of the cleaning device 100 without the ozone-generating light source 122 or those germicidal-only cleaning cycles in which the ozone-generating light source 122 is not operated, the germicidal time period may be configured to provide the germicidal effect without regard to depletion of the ozone (e.g., being less than one minute).

In those embodiments of the cleaning device 100 that include the ozone-dissociating light source 126, the germicidal time period may be shorter due to the cumulative ozone-dissociating effect of the UVC germicidal light source 124 and the ozone-dissociating light source 126. Alternatively, the germicidal light time period may be configured to provide the germicidal effect without regard to depletion of the ozone, which is instead performed by the ozone-dissociating light source 126.

In still further examples, for those cleaning devices 100 that include a sensor 150 configured to detect ozone, the UVC germicidal light source 124 may be operated until the level of ozone detected by the sensor 150 reaches a predetermined concentration (e.g., substantially 0 ppm), or until achieving the later of the UVC germicidal time period or the predetermined concentration of ozone.

For those embodiments of the cleaning device 100 that include the ozone-dissociating light source 126, the ozone-dissociating time period is configured for the ozone-dissociating light source 126 to substantially deplete the ozone, as described above to substantially 0 ppm. The ozone-dissociating light emitted by the ozone-dissociating light source 126 may dissociate ozone at a faster rate than the UVC germicidal light emitted by the UVC germicidal light source 124. To substantially deplete the ozone, the ozone-dissociating time period terminates after and is longer than the ozone-generating time period. The ozone-dissociating time may be started simultaneous with the ozone-generating time period or thereafter (e.g., before or after termination of the ozone-generation time period).

In the ozone cleaning cycle described above for achieving between 2 ppm and 10 ppm of ozone, the ozone-dissociating time period may, for example, be between three and twelve minutes, such as between five and eight minutes, more or less. The UVC germicidal light source 124 may, as referenced above, be operated concurrently with the ozone-dissociating light source 126 for cumulative ozone-dissociating effect, for example, with the germicidal time period being equal to the ozone-dissociating time period. In other cleaning cycles (e.g., for those having longer ozone-generating time periods or higher concentrations of ozone), the ozone-dissociating time period may be longer.

In another example, for those cleaning devices 100 that include the sensor 150 configured to detect ozone, the ozone-dissociating light source 126 may be operated, instead of or in addition to the ozone dissociating time period, until the level of ozone detected by the sensor 150 reaches a predetermined concentration (e.g., substantially 0 ppm) or the later of achieving the ozone-dissociating time period and the predetermined concentration of ozone.

For those embodiments of the cleaning device 100 that include the blue germicidal light source 128, the blue germicidal time period is configured for the blue germicidal light source 128 to support the germicidal effect of the UVC germicidal light source 124. The blue germicidal time period may, for example, be concurrent with and equal to the UVC germicidal time period.

The agitator 130 is operated for an agitator time period, which may equal to the total time of the entire cleaning cycle, such as by equaling the UVC germicidal time period and/or the ozone-dissociating time period and any portions of the ozone-generating time period occurring therefore and the scent time period thereafter. As such, the agitator 130 may support the germicidal effect (e.g., by moving the articles to help ensure receipt of the UVC germicidal light thereon) and/or ozone depletion (e.g., by circulating the ozone to receive the UVC germicidal light and/or the ozone dissociating light thereof).

For those embodiments of the cleaning device 100 that include the scent emitter 160, the scent time period is configured for the scent emitter 160 to emit the scent to be absorbed by the articles or other desired criteria. The scent emitter 160 may, for example, be operated for a scent time period of between 1 second and 60 seconds (e.g., 15 seconds) or other amount of time, to emit sufficient fragrance for a desired effect. The scent time period may be at the end or after the UVC germicidal time period, the ozone-dissociating time period, and/or the blue germicidal time period. The scent time period may coincide with the agitator time period, such that the agitator 130 (e.g., a fan) is operated while the scent emitter 160 is operated to circulate the fragrance within the chamber 214.

In one specific example, a predetermined ozone cleaning cycle is started upon receiving a user input (e.g., a button press). The control system 140 simultaneously starts operating the ozone-generating light source 122, the UVC germicidal light source 124, and the agitator 130 (i.e., a fan). The control system 140 continues operating the ozone-generating light source 122 for the duration of the ozone-generation time period (e.g., 30 seconds), which may be to generate a desired amount or concentration of ozone (e.g., 8 ppm). Upon terminating operation of the ozone-generating light source 122 (i.e., upon completion of the ozone-generation time period), the control system 140 continues to operate the UVC germicidal light source 124 for a remainder of the UVC germicidal time period (e.g., 10 minutes) and the agitator 130 for a remainder of the agitator time period (e.g., 10 minutes), which may be to substantially deplete the ozone.

In a variation of the predetermined ozone cleaning cycle, the ozone-generating time period is longer (e.g., two and five minutes), which may be to generate a greater amount or concentration of ozone (e.g., between 40 ppm and 80 ppm). The UVC germicidal time period is longer (e.g., between 40 minutes and 120 minutes), which may be to substantially deplete the ozone.

In a further variation of the predetermined cleaning cycle, the control system 140 further includes starting operation of the ozone-dissociating light source 126 simultaneous with the ozone-generating light source 122, the UVC germicidal light source 124, and the agitator 130. Upon terminating operation of the ozone-generating light source 122, the control system 140 additionally continues to operate the ozone-dissociating light source 126 for a remainder of the ozone-dissociating time period, which may be to substantially deplete the ozone and equal to the germicidal time period and the agitator time period.

In another example, a user provides an input to select and initiate one of multiple different cleaning cycles. For example, the cleaning device 100 may be configured to provide two or more cleaning cycles, which may include a first ozone cleaning cycle (e.g., to achieve an ozone concentration of 10 ppm or below), a second ozone cleaning cycle (e.g., to achieve an ozone concentration of between 10 ppm and 60 ppm), and/or a UVC germicidal cleaning cycle (e.g., without any ozone generation). The first ozone cleaning cycle and the second ozone cleaning cycle are differentiated by the second cleaning cycle having longer operational time periods (e.g., between five and twenty times longer than those of the first ozone cleaning cycle).

Referring to FIG. 3, the method 300 for cleaning articles with a cleaning device includes receiving articles 310 in an enclosure, receiving a user input 320 to perform a cleaning cycle, and performing the cleaning cycle 330. The performing of the cleaning cycle 330 includes operating an ozone-generating light source 340, operating a UVC germicidal light source 350, and operating an agitator 360. The performing of the cleaning cycle 330 may further include one or more of operating an ozone-dissociating light source 370, operating a blue germicidal light source 380, or operating a scent emitter 390.

The receiving of the articles 310 is performed with the enclosure, such as the enclosure 110. The receiving of the articles 310 may include a user or machine placing the articles in the chamber 214, for example, to be supported by the article support 219 (e.g., being suspended thereby or resting thereon).

The receiving of the user input 320 is performed with a control system, such as with the user input 142 in communication with the control system 140. In one example, the user input is binary input to starting the cleaning cycle (e.g., a button press), while in other examples, the user input may be a user selection from multiple different cleaning cycles to be started, or may be or include different inputs (e.g., types of articles, level of clean requested, cleaning duration, ozone level) from which the control system determines or selects a cleaning cycle.

The performing of the cleaning cycle 330 is controlled by the control system to perform the operating of the ozone-generating light source 340, the operating of the UVC germicidal light source 350, and the operating of the agitator 360, and may further include one or more of the operating of the ozone-dissociating light source 370, the operating of the blue germicidal light source 380, or the operating of the scent emitter 390.

The operating of the ozone-generating light source 340 includes operating the ozone-generating light source for an ozone-generating time period with the control system. The ozone-generating light source may be the ozone-generating light source 122 described previously, which is operated to emit ozone-generating light (e.g., with a peak wavelength of 185 nm). The ozone-generating time period may be as described above.

The operating of the UVC germicidal light source 350 includes operating the UVC germicidal light source for a UVC germicidal time period with the control system. The UVC germicidal light source may be the UVC germicidal light source 124, which is operated to emit UVC light with germicidal effect (e.g., with a peak wavelength of 254 nm). The UVC germicidal time period time period may be as described above.

The operating of the UVC germicidal light source 350 may be performed in parallel with the operating of the ozone-generating light source 340 (e.g., being started substantially simultaneously with the ozone-generating light source, as represented by the solid arrow in FIG. 3) or serially after starting or completing the operating of the ozone-generating light source 340 (e.g., as indicated by the dashed arrow in FIG. 3). The term “substantial” as used with respect to time periods may be an absolute term (e.g., within 5, 3, 2, or 1 second or less being substantially simultaneous) or a relative term (e.g., within 5%, 3%, 2%, or 1% or less being substantially equal).

The operating of the agitator 360 includes operating the agitator for an agitator time period with the control system. The agitator may be the agitator 130, as described above, such as a fan that circulates gases in the enclosure and/or moves the articles within the enclosure. The agitator time period, as described above, be substantially equal to the total time of the entire cleaning cycle, substantially equal to the UVC germicidal time period, or substantially equal to the longer of the UVC germicidal time period or the ozone-dissociating time period.

The operating of the agitator 360 may be performed in parallel with the operating of the ozone-generating light source 340 (e.g., being started substantially simultaneous with the ozone-generating light source) or serially after the operating of the ozone-generating light source 340 is started. The operating of the agitator 360 may further be performed in parallel with the operating of the UVC germicidal light source 350, for example, being started and terminating substantially simultaneously therewith.

The operating of the ozone-dissociating light source 370 includes operating the ozone-dissociating light source for an ozone-dissociating time period with the control system. The ozone-dissociating light source may be the ozone-dissociating light source 126 described previously (e.g., operating with a peak output of 290 nm). The ozone-dissociating time period may be as described above (e.g., equal to the UVC germicidal time period. The operating of the ozone-dissociating light source 370 may be performed in parallel with the operating of the UVC germicidal light source 350, for example, being started and terminating substantially simultaneously therewith.

The operating of the blue germicidal light source 380 includes operating a blue germicidal light source for a blue germicidal time period with the control system. The blue germicidal light source may be the blue germicidal light source 128 described previously (e.g., operating with a peak output of between 400 nm and 440 nm). The blue germicidal time period, as described above may be as described above (e.g., equal to the UVC germicidal time period. The operating of the blue germicidal light source 380 may be performed in parallel with the operating of the UVC germicidal light source 350, for example, being started and terminating substantially simultaneously therewith.

The operating of the scent emitter 390 includes operating a scent emitter for a scent time period with the control system. The scent emitter may be the scent emitter 160 described previously. The scent time period, as described above, may be relatively short as compared to and performed at the end of the agitator time period (e.g., terminating substantially simultaneous with the operating of the agitator 360).

Referring to FIGS. 4A-4I, the cleaning device 100 generally includes the enclosure 110, various of the light sources 120, the agitator 130, and the control system 140, and may further include the sensors 150 and/or the scent emitter 160, as described previously and as described in further detail below.

The enclosure 110 includes the body 212, which has a configuration similar to the shell 620 of the device 600 described below. The body 212 has a rectilinear shape with four upright sides 412a-d, an upper side 412e, and a lower side 412f that cooperatively define the chamber 214 therebetween. A front side 412a defines the opening 216 and includes the closure 218, which is configured as a door that is movable between a closed configuration and an open configuration.

The four upright sides 412a-d each have an inner surface 412g and an outer surface 412h. The inner surface 412g is reflective, such that the light emitted by the various of the light sources 120 reflects off of the inner surfaces 412g to impinge on the articles from different angles. The upper side 412e and/or the lower side 412f may also inner surfaces 412g that are reflective.

In one example, the inner surfaces 412g are formed by a material that is reflective to the UVC germicidal light, which may be a flexible film, sheet material, or rigid and polished surface. For example, the inner surfaces 412g may be formed of metal and/or polymer materials, such as aluminum, a metallized biaxially-oriented polyethylene terephthalate (e.g., Mylar®, as described below for the third layer 628 of the shell 620), or other metallized polymer. For example, as shown in FIG. 4C with respect to the right side 412b, the upright sides 412a-d may each have a layered configuration that includes an inner layer 414 and an outer layer 416, and may further include one or more intermediate layers 418 arranged therebetween. The inner layer 414, may be formed of the sheet material as described above, to form the inner surface 412g thereof. The outer layer 416 forms the outer surfaces 412h of the upright sides 412a-d and may be formed of a material that is more durable to contact and handling than that inner layer 414 and/or any of the intermediate layers 418. The outer layer 416 may, for example, be a flexible sheet material (e.g., a woven textile, such as nylon or canvas, or extruded polymer sheet), as described below for the first layer 624 of the shell 620. Individually or cooperatively, the various layers function to substantially seal the chamber 214 from exfiltration (e.g., from ozone leaving the chamber 214) and leakage of some or all light (e.g., ultraviolet light) from the chamber 214. One of the upright sides 412a-d or the closure 218 may further include a window, which allows visible light to pass therethrough but which filters (e.g., absorbs) ultraviolet from leaving the chamber 214. The upper side 412e and/or the lower side 412f may be configured as described for the upright sides 412a-d (e.g., by having a flexible film or sheet material and/or a layered configuration).

Referring to FIGS. 4B and 4C, the upright sides 412a-d may be flexible, for example by being formed with the flexible sheet materials described previously, and be supported by a frame 420 (e.g., similar to the frame 610). The frame 420 may include elongated frame members 420a that extend vertically and/or horizontally and are sufficiently rigid to support the flexible sheet materials forming the sides 412a-f. For example, as shown, the frame 420 includes four vertical elongated frame members and four horizontal elongated frame members, which are arranged in a rectilinear shape (e.g., proximate corners between the sides 412a-f). In those embodiments in which the front side 412a is flexible, the closure 218 may seal the chamber 214 by removably coupling to the front side 412a with a zipper, hook and loop fastener, or other coupling mechanism (e.g., as described below with respect to the door 629). For example, an outer periphery of the closure 218 and an inner periphery of the opening 216 of the front side 412a may include mating portions of such a coupling mechanism. Such a coupling mechanism may further include a gasket or other type of seal.

The frame 420 is coupled to or otherwise engages the upright sides 412a-d and/or the upper side 412e and the lower side 412f to provide the overall shape to the enclosure 110. The frame 420 and/or the elongated frame members 420a thereof may be positioned within the sides 412a-f (e.g., between the inner layer 414 and the outer layer 416 thereof, as shown and represented by being illustrated in dashed lines), inside the chamber 214, outside the chamber, or between the sides 412a-f (e.g., being an intermediate member to which the sides 412a-f are coupled). The frame 420 may be arranged in any other suitable manner, for example, with each of the sides 412a-f having four elongated members (e.g., at the edges thereof) and/or diagonal members (e.g., extending across various of the sides 412a-f). The frame 420 (e.g., the frame members 420a thereof) may be made from any suitable material to support the sides 412a-f to define the chamber 214 and/or to support the article support 219 and any articles further supported thereby, such as polymers, fiber-reinforced polymers, metal, or other suitable material.

As an alternative to the sides 412a-f being flexible and supported by the frame 420, one or more of the sides 412a-f may be rigid (e.g., all such sides, only the lower side 412f, or any other suitable combination). The sides 412a-f may be formed of any suitable material or combinations of materials, such as with rigid panels formed of aluminum in a monolithic or layered configuration. The inner surfaces 412g are reflective, as described above, and may be formed of a sheet material, as described above, or other suitable material (e.g., aluminum of appropriate surface finish). The closure 218 may be a rigid door that pivots about a hinged end, while a swinging end is releasably coupleable to the front side 412a with a latch or other mechanism. A seal (e.g., a gasket) is compressed between the closure 218 and the front side 412a (e.g., extending around the opening 216) to prevent exfiltration of gas (e.g., ozone) between the closure 218 and the front side 412a.

Referring to FIGS. 4A and 4C, each of the light sources 120 is arranged within the chamber 214. For example, as shown, one of the light sources 120 is positioned proximate one or more of the corners of the body 212 that are between two of the upright sides 412a-d. For example, as referenced above, the light sources 120 may each be a mercury lamp or otherwise have a tubular structure with an axis that extends parallel with the corner of the body 212 adjacent thereto (e.g., formed by edges of the upright sides 412a-d).

The light sources 120 may be coupled to the enclosure 110 in any suitable manner, for example, being coupled to the upright sides 412a-d, the upper side 412e, or the frame 420. The light sources 120 may be fixedly coupled in a singular location and orientation relative to the enclosure 110, or may in other embodiments be movable within the enclosure 110.

Referring to FIG. 5, one or more of the light sources 120 may be movable within the chamber 214. For example, the light sources 120 may include one or more of the UVC germicidal light sources 124, one or more of which (e.g., all) are movable within the chamber 214. Each of the UVC germicidal light sources 124 that are movable may be coupled to one end of a cord 524a by which power is transferred to the UVC germicidal light source 124, while another end of the cord 524a is fixedly coupled to another portion of the cleaning device 100 (e.g., to a power source). The cord 524a is flexible and of sufficient length for the UVC germicidal light source 124 to be moved about the chamber 214 to indeterminant locations (shown in solid lines), for example, to be inserted into articles to be cleaned (e.g., shoes, or other articles defining recesses or voids). The UVC germicidal light sources 124 are removably couplable to one or more coupling locations 524b at which the UVC germicidal light sources 124 are removably held (shown in dashed lines) in a predetermined position and/or orientation relative to the enclosure 110. The cleaning device 100 may include two of the UVC germicidal light sources 124 with coupling locations 524b arranged toward an upper end of the enclosure 110 and toward a lower end of the enclosure 110. The cleaning device 100 may also include one or more others of the light sources 120, such as the ozone-generating light source 122, which may be fixedly coupled inside the chamber 214 (e.g., being arranged toward the upper end thereof, as shown). The cord 524a may instead be configured as an articulating or bendable arm that supports the UVC germicidal light source 124 in variable locations within the chamber 214.

Referring again to FIGS. 4A and 4C, the agitator 130 may be arranged in a lower portion of the chamber 214, for example, being coupled to or otherwise proximate the lower side 412f of the body 212. When configurated as a fan, the agitator 130 may circulate the gas in the chamber 214, including ozone which has a higher density than ambient air and will tend to sink within the chamber 214. Thereby, the agitator 130 may circulate the ozone throughout the chamber 214 to penetrate the articles being cleaned (e.g., the fabric of clothing articles) and for the UVC germicidal light and/or the ozone-dissociating light to impinge on and, thereby, dissociate the ozone being circulated.

The agitator 130 may be arranged proximate a corner between the upright sides (e.g., between the left side 412c and the rear side 412d, as shown), biased toward one side but positioned centrally between sides perpendicular thereto (e.g., being biased toward the rear side 412d, while centrally-located between the left side 412c and the right side 412b), or be centrally-located between all of the upright sides 412a-d.

The article support 219 is positioned within the chamber 214 to support articles to be cleaned by ozone, the UVC germicidal light, and/or the blue germicidal light. The article support 219 may be configured in various manners, for example, including one or more of a hanging rod (see FIGS. 2B, 4B, and 4D), a shelf (see FIGS. 4E and 4F), a rack (see FIGS. 4G and 4H), or hanging lines (see FIGS. 4I and 4J), as discussed in further detail below. The various configurations of the article support 219 support the articles spaced apart from the sides 412a-f, such that the ozone and/or the germicidal light reaches the article. The article support 219 may, itself, be supported in the chamber 214 of the enclosure 110 in any suitable manner, for example, being coupled to the frame 420 (e.g., in the case of various of the sides 412a-f being flexible), the sides 412a-f (e.g., in the case of the sides 412a-f being rigid), and/or to rest on the lower side 412f or structure therebetween. In FIGS. 4B and 4D-4I, the light sources 120 are omitted from the drawings for the ease of illustration, but it should be understood that various configurations of the article support 219 are compatible with and used in conjunction with the light sources 120 (e.g., being spaced apart therefrom).

As illustrated in FIGS. 4B and 4D, the article support 219 is configured as a hanging rod 419a. As shown, the hanging rod 419a is coupled to the frame 420 and supported thereby proximate an upper end of the chamber 214, but may instead be coupled to various of the sides 412a-d or the upper side 412e (e.g., if formed of rigid material). The hanging rod 419a is configured to receive thereon and suspend therefrom clothing hangers (not shown) from which clothing or other articles may be suspended. Various of the clothing hangers may, for example, have a width that separates layers of fabric of clothing articles hanging therefrom (e.g., front and back fabric or a shirt). Others of the clothing hangers include spring clips or hooks from which smaller articles may be suspended, such as socks or undergarments. Flexible articles suspended by the hanging rod 419a are moved by the agitator 130, for example, moving with the air flow from the fan or being physical engaged by the agitator 130.

As illustrated in FIGS. 4E and 4F, the article support 219 is configured as a shelf 419b. The shelf 419b is configured to permit the ozone and/or germicidal light to pass therethrough to reach an article positioned thereon. In one example, the shelf 419b may be formed of a quartz material that allows the germicidal light to pass therethrough to impinge on that surface of the article resting on the shelf 419b (e.g., a laptop computer). In another example, the shelf 419b may be formed of a highly porous material, such as a mesh (e.g., formed of a rigid material or woven material in tension), which allows ozone to pass therethrough and may further allow germicidal light to pass therethrough. The shelf 419b may, as shown, be configured to move from a stored position (shown in dash-dot lines) to a use position (shown in solid lines). For example, the shelf 419b may be hingedly coupled to the frame 420 and/or to one or more of the upright sides 412a-d.

As illustrated in FIGS. 4G and 4H, the article support 219 is configured as a rack 419c. The rack 419c is configured to suspend the articles above the lower side 412f of the enclosure 110. For example, the rack 419c may include a series of elongated members (e.g., rods or bars) over which one or more articles may be draped. For example, the rack 419c may be configured to support wet clothing articles (e.g., from a washing machine), which provides the further benefit to conventional washing by further cleaning the article with ozone and/or germicidal light to prevent generation of mold and/or mildew. The rack 419c may, as shown, be configured to move from a stored position (shown in dash-dot lines) to a use position (shown in solid lines). For example, the rack 419c may be hingedly coupled to the frame 420 and/or to one or more of the upright sides 412a-d. The lower side 412f may further be configured to capture water that may drip from wet articles on the rack 419c, while providing an outlet for releasing water therefrom (e.g., a plug), or alternatively, a removable water tray 417 may be inserted into the chamber 214 and rest on the lower side 412f The rack 419c may be configured in other manners, for example, being removable and/or resting on the lower side 412f (e.g., for supporting a laundry basket thereon with the agitator 130 (e.g., a fan) directed theretoward.

As illustrated in FIGS. 4I and 4J, the article support 219 includes a plurality of lines 419d. Each of the lines 419d is suspended and hangs downward from an upper portion of the enclosure 110, such as the frame 420, the upper side 412e (as shown), or a hanging rod (e.g., such as the hanging rod 419a). Each of the lines 419d includes one or more (e.g., 5, more or less) attachment devices 419d′, such as a hook, clasp, or clip (e.g., a spring clip), that are configured to removably couple to articles to be cleaned by the cleaning device 100. For example, the attachment devices 419d′ may be configured to couple to and support face masks (illustrated; not labeled) used in a medical facility (e.g., hospital). In FIG. 4I one of the attachment devices 419d′ is illustrated schematically, but it should be understood that one of the attachment devices 419d′ corresponds to each of the articles (e.g., masks) supported on the lines 419d. The lines 419d may be flexible, such that the lines 419d and the articles coupled thereto are moved by the agitator 130 within the chamber 214. The line 419d may, for example, be formed by a string, wire, chain, or other elongated and flexible material or structure. Instead, the lines 419d may be generally rigid structures, while the attachment devices 419d′ are configured to move relative thereto or to allow the articles to move relative thereto (e.g., attaching to flexible portions of the article, such as a strap of the facemask).

The lines 419d and the attachment devices 419d′ thereon are configured for the articles, such as masks, to be spaced apart from each other. Spacing apart the articles may help ensure that the articles have limited physical contact with each other and do not prevent the UVC germicidal light from impinging on others of the articles, directly from the light sources 120 or indirectly by reflecting off the inner surfaces 412g of the body 212.

For example, the attachment devices 419d′ may be spaced apart along the lines 419d from each other a distance of between 6 inches and 18 inches, such as between 6 inches and 12 inches (e.g., between 6 inches and 9 inches), which may be referred to as vertical spacing. The vertical spacing may be specifically configured and account for the articles being medical masks having both a height and a width of 5 inches to 6 inches and a smaller depth (e.g., 2 inches to 3 inches). Further, while illustrated with the attachment devices 419d′ supporting the face masks at the same heights, the attachment devices 419d′ may be at staggered heights relative to adjacent ones of the lines 419d to facilitate light impinging on each of the masks in a horizontal plane.

Alternatively, the vertical spacing may be adjustable (e.g., with the attachment devices 419d′ being movably coupleable to the lines 419d.

The lines 419d may be supported within the chamber 214 of the enclosure 110 (e.g., being coupled to another article support 219, the frame 420, the upper side 412e, or another structure) at spaced apart locations, which may be referred to as lateral or horizontal spacing. The lateral spacing of the lines 419d may, for example, be between 6 inches and 12 inches. It should be understood that because the lines 419d are flexible lower portions of the lines 419d may have variable spacing therebetween from the force of articles hanging therefrom and movement caused by the agitator 130. Further arrangements are contemplated in which the lines 419d are arranged relative to each other while providing clear line of sight in at least two directions perpendicular to the upright sides 412a-d from the articles (e.g., masks) to the upright sides 412a-d (e.g., four lines 419d in a square or rectilinear arrangement, three or more lines 419d arranged diagonally relative to the upper sides 412a-d, a crossing pattern (e.g., five of the lines 419d arranged in an X-pattern). Alternatively, the quantity and/or lateral spacing of the lines 419d may instead be adjustably (e.g., removable from and/or movable along the hanging rod 419a).

While the article supports 219 of the hanging rod 419a, the shelf 419b, the rack 419c, and the lines 419d are illustrated independent of each other in FIGS. 4A-4J, the cleaning device 100 may be configured to itself include two or more of the article supports 219 or be provided as a system that includes two or more of the article supports 219. In one example, the cleaning device includes both the shelf 419b and the rack 419c, which as illustrated in FIGS. 4E to 4H, pivot from different ones of the upright sides 412a-d of the body 212 (e.g., the left side 412c and the right side 412b). In another example, the cleaning device 100 includes the hanging rod 419a along with shelf 419b and/or the rack 419c with the hanging rod 419a being positioned above the shelf 419b and/or the rack 419c to permit the pivoting thereof from the respective stowed positions to use positions. In a still further example, the cleaning device 100 may be provided as a system that includes one or more of the hanging rod 419a, the shelf 419b, the rack, 419c, and/or the lines 419d that are removably coupleable to an inner support structure (e.g., the frame 420 or coupling locations on the sides 412a-f).

As shown in FIGS. 6 to 8, an embodiment of a cleaning device 600 is provided for articles such as clothing. The cleaning device 600 preferably comprises a frame 610, a shell 620, an ultraviolet light 630, and a control system 640. The ultraviolet light 630 preferably emits UVC light. In device 600, UVC light operates as the cleanser for articles provided within the device 600.

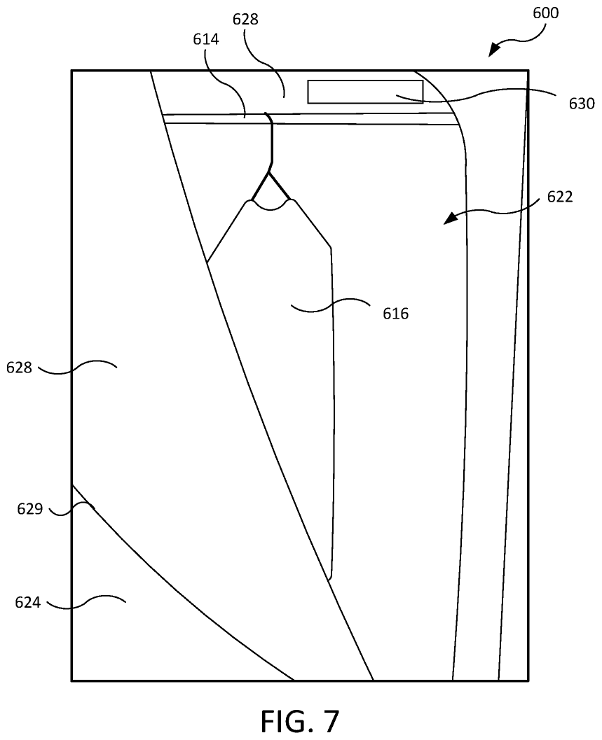

The frame 610 preferably comprises of a plurality of interconnected frame rods 612 (hidden; illustrated in FIG. 6 in dash-dash lines). The frame 610 is configured to provide the device 600 in a generally rectangular cuboid shape that will rest on a surface, such as a floor or table. The frame 610 may be hidden from view, for example, being arranged within the interior and/or between the layers of the shell (discussed below). Alternative shapes for the device 600 are contemplated, such as a cylindrical shape, or other suitable shape to be appreciated by those of skill in the art. The overall size of the frame 610, and hence the device 600, is variable, but it will typically be the size of a pop-up closet for apparel. Smaller sizes for personal traveling and portable use, as well as much larger sizes for commercial, industrial, and/or institutional use are contemplated. In a preferred embodiment for residential use, the frame 610 is preferably collapsible, such that the device 600 may be stowed away when not in use. It is contemplated that the frame 610 may comprise a unitary and permanent construction. The frame 610 further comprises a hanging rod 614, wherein the hanging rod 614 and the frame 610 are configured to support the weight of several articles 616, such as household objects including clothing, that are provided on the hanging rod 614. See FIG. 7. The hanging rods 614 are preferably formed of aluminum, plastic, or other durable material suitable for use in the device 600.

The shell 620 is preferably provided over the frame 610, thereby defining an interior 622 of device 600. The shell 620 formed of a plurality of layers. A first layer 624 of the shell 620 is preferably formed of a durable material for the outermost protection of the cleaning device 600. A second layer (not labeled) of the shell 620 is preferably formed of an insulating material that is impenetrable by UVC and other wavelengths of light. Suitable materials forming the second layer include black nylon and black canvas. A third layer 628 of the shell 620 is preferably formed of a material suitable for the reflection of UVC, such as aluminum and/or Mylar® film. As shown in FIG. 6, the shell preferably comprises a door 629 through which articles 616 may be provide in and retrieved from the interior 622. The door 629 may be provided with a closure mechanism, such as a zipper, hook and loop strips, or other suitable fastening means.

At least one of the ultraviolet lights 630 is provided within the interior 622 of device 600. In a preferred embodiment of the present invention, a plurality of the ultraviolet lights 630 may be used. The ultraviolet lights 630 emit UVC light, and are strategically positioned in within the interior 622 to provide suitable exposure of the UVC light to a preferably complete surface area of articles 616 placed in device 600 for cleaning. The one or more ultraviolet lights 630 are preferably encased in a quartz housing (not separately identified) and connected to the control system 640 and an electrical power source, such as an electrical outlet or portable power source.

The control system 640 preferably comprises one or more power source connectors, switches, processors, data storage means, timers, audio mechanisms, video mechanisms, user interfaces, and communication means, such as wifi and short-range wireless communications technology (i.e., Bluetooth® technology). In the example, shown, the control system 640 includes a plug for connecting to a power source (e.g., the electrical outlet). It is contemplated that device 600 may communicate with smartphones, smarthomes, and related technologies. Switches may be used to control the ultraviolet lights 630. For safety and efficiency, the control system 640 is configured to monitor the door 629 of device 600, to ensure that the ultraviolet lights 630 are activated only when the door 629 is completely closed such that a user of the device 600 is not inadvertently exposed to the UVC light. The control system 640 also monitors the device 600 to ensure that the ultraviolet lights 630 refrain from operating unless articles are provided within the device 600.

Some alternative preferred embodiments of device 600 may comprise a means for selectively introducing an odor eliminating substance into the interior 622.

As shown in FIGS. 9 and 10, alternative preferred embodiments of the present invention are provided.

As shown in FIG. 9, an alternative device 900 comprises a base 914, a shell 920, an ultraviolet light (not shown), a control system (not shown; see the control systems 640 and 1040), a shaft 960, a motor 970. The shell 920 is illustrated in FIG. 9 in a partial view with two sides thereof removed in order to better illustrate features of the device 900 internal to the shell 920 (e.g., the base 914, the shaft 960, and the motor 970).

The shell 920 is preferably formed of a material that is impenetrable by UVC light, such as a durable plastic, and lined with aluminum or other suitable reflective material. The shell 920 preferably further comprises a lid 921. The ultraviolet light (not shown) may be connected to the lid 921 (e.g., as shown for the ultraviolet light 1030 in FIG. 10), the base 914, the shaft 960, or the interior surface 923 of shell 920. The base 914 is preferably connected to the shaft 960 and configured to be driven by the motor 970 to move about the shaft 960. With articles (not shown) for cleaning, such as clothing, are positioned on the base 914, the aforementioned movement about the shaft 960 will serve to “randomize” the articles such that the surface area of the articles is completely exposed to the UVC light emitted by the ultraviolet light during a cleaning process using the device 900. It is contemplated that movement of the base 914 about the shaft 960 may be smooth, intermittent, abrupt, or a combination thereof, such that the position of the articles is suitably manipulated for cleaning.

As shown in FIG. 10, an alternative device 1000 comprises a base (not shown), a shell 1020, an ultraviolet light 1030, a control system 1040, a shaft 1060, and a motor (not shown).

The shell 1020 is preferably formed of a material that is impenetrable by UVC light, such as a durable plastic or aluminum, and lined with aluminum or other suitable reflective material (if the shell 1020 is not formed of aluminum). The shell 1020 preferably further comprises a lid 1021. The ultraviolet light 1030 may be connected to the lid 1021, the base, the shaft 1060, or an interior surface 1023 of the shell 1020. The base is preferably connected to the shaft 1060 and configured to be driven by the motor to move about the shaft 1060 (e.g., as shown and described for the base 914, the shaft 960, and the motor 970). Wherein articles (not shown) for cleaning, such as clothing, are positioned on the base, the aforementioned movement about the shaft 1060 will serve to “randomize” the articles such that the surface area of the articles is completely exposed to the UVC light emitted by the ultraviolet light 1030 during a cleaning process using device 1000. It is contemplated that movement of the base about the shaft 1060 may be smooth, intermittent, abrupt, or a combination thereof, such that the position of the articles is suitably manipulated for cleaning. The motor may drive the base to rotate around the shaft 1060 or move up and down about the shaft 1060 or both.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A cleaning device comprising:
an enclosure;
an ozone-generating light source configured to generate ozone in the enclosure;
a UVC germicidal light source configured to emit UVC germicidal light in the enclosure; and
a control system configured to, during a predetermined cleaning cycle, operate the ozone-generating light source for a first time period and to operate the UVC germicidal light source for a second time period, the second time period being at least 10 times longer than the first time period and terminating after the first time period.

2. The cleaning device according to claim 1, further comprising a fan that is operated by the control system during the predetermined cleaning cycle;
wherein the first time period is between 10 seconds and 1 minute and is configured to generate between 2.5 ppm and 10 ppm of ozone in the enclosure if the enclosure were to not contain articles to be cleaned;
wherein the second time period is between 5 minutes and 20 minutes, is configured relative to the first time period to substantially deplete the ozone in the enclosure, and starts one of simultaneous with the first time period or after starting the first time period.

3. The cleaning device according to claim 1, wherein the second time period is at most 30 times longer than the first time period.

4. The cleaning device according to claim 1, wherein the first time period is between 10 seconds and 1 minute

5. The cleaning device according to claim 4, wherein the second time period is between 5 minutes and 20 minutes.

6. The cleaning device according to claim 1, wherein during the predetermined cleaning cycle, the second time period starts one of simultaneous with or subsequent to starting of the first time period.

7. The cleaning device according to claim 1, wherein the first time period is configured for the ozone-generating light source to generate between 2.5 ppm and 10 ppm of ozone if the enclosure were to not contain articles to be cleaned.

8. The cleaning device according to claim 7, wherein the second time period configured relative to the first time period to substantially deplete the ozone in the enclosure.

9. The cleaning device according to claim 1, further comprising a fan configured to circulate gas within the enclosure, wherein the control system operates the fan during the predetermined cleaning cycle.

10. The cleaning device according to claim 1, further comprising an ozone-dissociating light source that emits ozone dissociating light, wherein the control system operates the ozone-dissociating light source during the predetermined cleaning cycle.

11. A cleaning device comprising:
an enclosure;
an ozone-generating light source configured to generate ozone in the enclosure;
a UVC germicidal light source configured to emit UVC germicidal light in the enclosure; and
an article support configured to suspend an article therefrom within the enclosure to be cleaned by the ozone and the UVC germicidal light.

12. The cleaning device according to claim 11, further comprising a fan;
wherein the article support includes one or more lines that are flexible suspended from an upper portion of the enclosure at lateral spacing of at least 6 inches;
wherein the article support further includes attachment devices that are configured to releasably couple to the articles to be cleaned and that are spaced apart along the lines at vertical spacing of at least 6 inches;
wherein the fan is operable to circulate gas within the enclosure to move the articles; and
wherein the enclosure includes four upright sides that are flexible, supported by a frame, and include an inner layer that forms reflects the UVC germicidal light in the enclosure.

13. The cleaning device according to claim 11, wherein the article support includes one or more lines that are flexible and suspended from an upper portion of the enclosure.

14. The cleaning device according to claim 13, wherein the article support further includes attachment devices that are configured to releasably couple to articles to be cleaned in the enclosure and that are spaced apart along the lines at vertical spacing of at least 6 inches.

15. The cleaning device according to claim 13, comprising two or more of the lines that are spaced apart along the upper portion at lateral spacing of at least 6 inches.

16. The cleaning device according to claim 13, further comprising a fan configured to circulate gas within the enclosure.

17. The cleaning device according to claim 11, wherein the enclosure includes interior surfaces that are reflective to the UVC germicidal light.

18. The cleaning device according to claim 17, wherein the enclosure includes four upright sides that are flexible and supported by a frame to define therebetween a chamber that is rectilinear.

19. The cleaning device according to claim 18, wherein the upright sides are formed of multiple layers of material, an inner layer forming the interior surfaces that are reflective to the UVC germicidal light.

20. The cleaning device according to claim 17, wherein the enclosure includes four upright sides that are rigid and define therebetween a chamber that is rectilinear.

21. A method for cleaning face masks with a cleaning device comprising:
receiving face masks in an enclosure of the cleaning device;
supporting the face masks in the enclosure with an article support of the cleaning device at predetermined locations on the support that are spaced apart by at least 6 inches;
generating ozone in the enclosure with an ozone-generating light source of the cleaning device; and
emitting UVC germicidal light in the enclosure with a UVC germicidal light source of the cleaning device.

22. The method of claim 21, wherein the generating of the ozone is performed by operating the ozone-generating light source for a first predetermined time period; and
wherein the emitting of the UVC germicidal light is performed by operating the UVC germicidal light source for a second predetermined time period configured for the ozone to be substantially depleted from the enclosure.

23. The method of claim 21, further comprising operating a fan to circulate the ozone and to move the face masks within the enclosure.
